(19) United States
(12) Patent Application Publication
(10) Pub. No.: US 2003/0229114 A1
(43) Pub. Date: Dec. 11, 2003

(54) Pyrroloquinoline quinone drugs as a neuroprotectant and methods of use thereof

(76) Inventors: Paul A. Rosenberg, Newton, MA (US); Yumin Zhang, Allston, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/407,698

(22) Filed: Apr. 4, 2003

Related U.S. Application Data
(60) Provisional application No. 60/370,459, filed on Apr. 4, 2002.

(51) Int. Cl. A61K 31/4745; A61K 9/127

(52) U.S. Cl. 514/291; 424/450

(57) ABSTRACT

The invention includes compositions comprising substantially purified pyrroloquinoline quinone, that are useful in methods for the treatment and prevention of acute and chronic neurological injury caused by peroxynitrite. The invention also includes methods for the treatment and prevention of neurological injury comprising contacting a composition of the invention with a human patient. In embodiments of the invention, pyrroloquinoline quinone is provided prior to formation of peroxynitrite.

### RELATED APPLICATIONS

This application claims the benefit of priority under 35 U.S.C. 119(e) to copending U.S. Provisional Application No. 60/370,459, filed Apr. 4, 2002, the entire contents of which are incorporated herein by reference.

### BACKGROUND OF THE INVENTION

Excessive or inappropriate generation of nitric oxide has been thought to be one of the major causes of a variety of acute neurological disorders, as well as chronic neurodegenerative diseases. In the central nervous system, nitric oxide is produced by neuronal nitric oxide synthase (nNOS), as well as by inducible NOS (iNOS) located in astrocytes and microglia. Nitric oxide is generally considered to be not toxic itself, but to react with superoxide to form peroxynitrite (ONOO⁻), a potent and toxic oxidant.

Nitric oxide has been implicated in NMDA receptor mediated neurotoxicity. In nNOS knockout mice, the area of infarction produced in a focal ischemia model was significantly less than in control animals of similar genetic background but with a normal nNOS gene, indicating that nitric oxide may play an important role in stroke. Later studies suggested that the neurotoxicity of nitric oxide in ischemic stroke depends upon its conversion to peroxynitrite.

### SUMMARY OF THE INVENTION

The present invention relates to the discovery that neurotoxicity can be prevented or minimized by administration of certain neuroprotective factors, and thus has benefit for treating acute and chronic neurological and other diseases. In particular, it has been found that non-toxic dosages of pyrroloquinoline quinone (“PQQ”) drugs are useful as neuroprotective agents, and are therefore valuable in the treatment of a variety of various acute and chronic neurological diseases such as stroke, amyotrophic lateral sclerosis (ALS, “Lou Gehrig's Disease”), Alzheimer's Disease, Huntington's Disease, and Parkinson's Disease. PQQ in particular has been found to modulate peroxynitrite-induced neurotoxicity such that neural cells are protected from cell death.

The compositions and methods of the invention are surprisingly useful for the reduction or elimination of peroxynitrite-induced neurological injury in vivo and ex vivo, as well as the prevention and/or treatment of acute and chronic neurological disease in a mammal in need thereof, such as humans.

The invention provides a method for preventing or inhibiting peroxynitrite-induced neurotoxicity in a mammalian subject in need thereof by administering to the subject an effective amount of a pyrroloquinoline quinone compound, where peroxynitrite is not present in toxic levels, e.g., at levels at or near normal physiological conditions, in the mammalian subject, such that peroxynitrite-induced neurotoxicity is prevented or inhibited. In embodiments of the invention, the pyrroloquinoline quinone is administered at a concentration between about 1 nM and about 500 μM. In some embodiments of the invention, the pyrroloquinoline quinone is administered at a concentration between about 1 nM and less than about 100 μM. In other embodiments, the pyrroloquinoline quinone is administered at a concentration between about 1 μM and about 100 μM. In other embodiments, the pyrroloquinoline quinone is administered at a concentration between about 100 μM and about 500 μM. In certain embodiments of the invention, the mammalian subject suffers from or is at risk of amyotrophic lateral sclerosis (ALS), and the method treats neurological manifestations of ALS.

The invention also provides a method for preventing, inhibiting or reducing neurological damage incident to a stroke in a mammalian subject at risk thereof by administering to the subject an effective amount of a pyrroloquinoline quinone compound prior to a measurable increase in peroxynitrite formation in the mammalian subject, such that neurological damage incident to stroke is prevented, inhibited or reduced.

The invention further provides a method for treating a chronic neurological disorder in a mammalian subject in need thereof by administering to the subject an effective amount of a pyrroloquinoline quinone compound at a point wherein peroxynitrite is not detectable at toxic levels, such that the chronic neurological disorder is reduced or eliminated.

The invention also provides a method for reducing endogenous peroxynitrite levels in a mammalian subject in need thereof, or in a cell, tissue or organ of the mammalian subject, by administering to the subject an effective amount of a pyrroloquinoline quinone compound, such that the endogenous peroxynitrite levels are reduced or at least maintained.

The invention provides a method for inducing cell toxicity in neural tissue by contacting said tissue with an effective amount of a pyrroloquinoline quinone compound that is capable of interacting with peroxynitrite, such that neural cell death increases in the neural tissue.

The invention further provides a method for preventing or inhibiting peroxynitrite-induced cell death in a mammalian subject at risk of or undergoing an acute traumatic neurological event by administering to the subject an effective amount of a pyrroloquinoline quinone compound at such time wherein peroxynitrite is not detectable at toxic levels, such that peroxynitrite-induced cell death is prevented or inhibited.

The invention also provides a kit for treating or preventing a neurotoxic injury, which includes in one or more containers a pyrroloquinoline quinone compound, a pharmaceutically acceptable carrier, and instructions for use of the kit.

Methods of treating neurotoxic injury are disclosed, wherein a pyrroloquinoline quinone compound and an antioxidant are administered to a subject at risk of an imminent neurotoxic injury caused by increased peroxynitrite levels, comprising administering, while peroxynitrite levels are not yet neurotoxic, a combination therapy including a PQQ compound and a second antioxidant, such that neurotoxic injury is treated or at least partially alleviated. The pyrroloquinoline quinone compound and antioxidant may be administered as part of a pharmaceutical composition, or as part of a combination therapy.

In another embodiment, a patient is diagnosed, e.g., to determine or measure if peroxynitrite levels are under such a level that treatment with the compound(s) of the invention will be potentially beneficial, whereupon a therapy or co-therapy in accordance with the invention is administered to treat the patient. The amount of pyrroloquinoline quinone compound and/or antioxidant is typically effective to reduce symptoms and to enable an observation of a reduction in symptoms.

These and other objects of the present invention will be apparent from the detailed description of the invention provided below.

### BRIEF DESCRIPTION OF THE DRAWINGS

FIG. 1 is a series of histograms and dose response graphs demonstrating that SIN-1 toxicity is mediated by peroxynitrite formation.

FIG. 1A is a dose response graph of neuronal cultures exposed to SIN-1 at selected concentrations up to 2 mM. After 24 hr, the reduction of Alamar Blue (AB) by living cells and the release of lactate dehydrogenase (LDH) from the dead cells were measured. The percentage survival of neurons measured by these two assays was the same. This experiment was repeated five times. In subsequent experiments, AB was used for assaying neurotoxicity.

FIG. 1B is a histogram demonstrating that SIN-1 toxicity is mediated by ONOO⁻ formation. SIN-1 (1 mM) toxicity was fully prevented by Hb (43 μM) or SOD (100 U/ml) together with catalase (CAT, 100 U/ml). A representative experiment of three that were performed is shown. **p<0.01 was obtained when the control and drug treated groups were compared.

FIG. 1C is a histogram demonstrating that MnTBAP (TBAP, 100 mM) completely blocks neurotoxicity induced by SIN-1 at 1 mM. A representative experiment of three that were performed is shown. ***p<0.001 was obtained when the control or TBAP treated group were compared.

FIG. 1D is a histogram demonstrating that MnTBAP fully protected against ONOO⁻ (100 μM) toxicity. A representative experiment of three that were performed is shown. ***p<0.001 was obtained when the control or TBAP treated group were compared.

FIG. 2 is a series of histograms and dose response graphs demonstrating that PQQ protects neural cells against SIN-1 but not peroxynitrite neurotoxicity.

FIG. 2A is a histogram showing that PQQ at 100 μM completely blocks neurotoxicity induced by SIN-1 (1 mM), ***p<0.001. Conversely, PQQ significantly potentiates the neurotoxicity induced by 100 μM ONOO⁻. The cell survival in this experiment was 52.4±0.4% and 11±8% without and with PQQ present, respectively. *p<0.05. A representative experiment of three that were performed is shown.

FIG. 2B is a dose response curve showing concentration dependence of PQQ protection against SIN-1 (1 mM) toxicity. A representative experiment of three that were performed is shown. The EC₅₀ of the protective effect of PQQ in this experiment was 22.6 mM.

FIG. 3 is a series of histograms and time course graphs demonstrating that PQQ blocks ATP depletion caused by SIN-1, but not by ONOO⁻.

FIGS. 3A and 3B demonstrate the time dependence of ATP depletion induced by 1 mM SIN-1 (FIG. 3A) or 100 mM ONOO⁻ (FIG. 3B). At the indicated time point, the cells were lysed and the intracellular levels of ATP were measured by firefly luciferase chemiluminescence. Experiments shown in A and B are representative of three that were performed.

FIG. 3C demonstrates that PQQ blocks depletion of ATP by SIN-1, but not ONOO⁻. At one hour following exposure of cultures to SIN-1 (1 mM) or ONOO⁻ (100 μM), the intracellular ATP was measured. PQQ at 100 μM significantly blocked ATP depletion induced by SIN-1 (93±12.6% compared to 43.3±18.7%, **p<0.01), but had no effect on ATP depletion caused by ONOO⁻ (43.8±4.7% compared to 37±9.9%, p>0.05). A representative experiment of three that were performed is shown.

FIG. 4 is a photograph of an immunoblot demonstrating the concentration dependence of the effect of PQQ on protein nitration caused by SIN-1 or ONOO⁻. BSA (0.5 mg/ml) in EBSS was exposed to SIN-1 (1 mM) or ONOO⁻ (100 μM) for one hr alone or in the presence of 100 μM, 300 μM or 500 μM PQQ. Samples were assayed by immunoblot analysis using an anti-nitrotyrosine monoclonal antibody. BSA nitration caused by SIN-1 was completely blocked by PQQ at all concentrations tested. Nitration of BSA by ONOO⁻ was increased by PQQ in a concentration-dependent manner. A representative experiment of three that were performed is shown.

FIG. 5 is a series of histograms and dose response graphs demonstrating that SIN-1 neurotoxicity, but not nitric oxide neurotoxicity, was independent of NMDA receptor activation.

FIG. 5A is a histogram showing that nitric oxide donors cause neurotoxicity mediated by activation of NMDA receptors. The neurotoxicity induced by three nitric oxide donors (DEA/NO, DPT/NO and DETA/NO), all at 1 mM, was examined in the absence or presence of 10 μM MK-801. Significant protection of MK-801 against neurotoxicity induced by each nitric oxide donor was observed. *p<0.05, ***p<0.001 were obtained when MK-801 treated groups were compared to the corresponding groups without addition of MK-801. A representative experiment of three that were performed is shown.

FIG. 5B is a dose response graph demonstrating that in contrast to what was observed with the nitric oxide donors in which MK-801 had an effect, MK-801 had no effect on SIN-1 toxicity. A representative experiment of three that were performed is shown.

FIG. 6 is a series of histograms demonstrating that PQQ has no effect on ATP depletion and neurotoxicity induced by nitric oxide donors.

FIG. 6A is a histogram showing that nitric oxide donors caused ATP depletion that was unaffected by the presence of PQQ. One hour after exposure to nitric oxide donors in the absence or presence of PQQ (100 μM), the intracellular ATP content was measured. DEA/NO or DPT/NO, both at 1 mM, caused reduction of ATP to 50.8±20.9% and 64.9±13.7% of control, respectively (*p<0.05, ***p<0.001). PQQ had no effect. In the presence of PQQ, the ATP levels were 42.9±19.6% and 72.7±7.9% in DEA/NO and DPT/NO groups, respectively. A representative experiment of three that were performed is shown.

FIG. 6B is a histogram showing that nitric oxide donors caused neurotoxicity that was unaffected by PQQ. Each of the nitric oxide donors at 1 mM caused significant neuronal death. The neuronal survival was 48.7±14.6%, 1.9±0.8%, and 25.2±10.6% of control in DEA/NO, DPT/NO, and DETA/NO groups, respectively (**p<0.01, ***p<0.001). PQQ at 100 μM showed no protective effect. In the presence of PQQ, the neuronal survival was 39.7±16.8%, 1.7±0.7%, and 30.7±7.7% of control, respectively. A representative experiment of three that were performed is shown.

FIG. 7 is a series of histograms demonstrating that PQQ and SOD both enhance cGMP accumulation.

FIG. 7A is a histogram demonstrating that PQQ enhanced cGMP accumulation induced by SIN-1, but not by ONOO⁻ or DEA/NO. Intracellular cGMP content was measured five minutes following exposure to SIN-1 (100 μM), ONOO⁻ (100 μM), or DEA/NO (10 μM), in the absence or presence of 100 μM PQQ. The cGMP content was 207.6±11.5%, 111±11.2%, and 215±32.2% of control in SIN-1, ONOO⁻ and DEA/NO treated groups, respectively, when PQQ was absent. In the presence of PQQ, the cGMP content was 313.5±45%, 142.5±16.2%, and 255±38% of control in SIN-1, ONOO⁻ and DEA/NO treated groups, respectively. *p<0.05, **p<0.01, ***p<0.001 were obtained when SIN-1 or DEA/NO groups with or without PQQ were compared to the control groups with or without PQQ, respectively. *p<0.05 was obtained when PQQ treated SIN-1 group was compared to SIN-1 group without addition of PQQ. A representative experiment of four that were performed is shown. In

FIG. 7B, SOD enhanced cGMP accumulation induced by SIN-1, but not by ONOO⁻ or DEA/NO. The experiment was performed as above. In the absence of SOD (100 U/ml), the cGMP content was 220.5±39.4%, 97±14.4%, and 318.5±31% of control in SIN-1, ONOO⁻, and DEA/NO treated groups, respectively. In the presence of SOD, the cGMP content was 327.7±39.5%, 131±6%, and 310.3±33.4% of control in the above groups, respectively. ***p<0.001 were obtained when the SIN-1 or DEA/NO groups with or without SOD were compared to the control groups with or without SOD, respectively. **p<0.01 was obtained when the SIN-1 group in the presence of SOD was compared to that in the absence of SOD. A representative experiment of four that were performed is shown.

FIG. 8 is a series of histograms and time course graphs demonstrating that the potentiation of SIN-1 toxicity by SOD is due to H₂O₂ production.

FIG. 8A is a histogram showing SOD potentiated SIN-1 toxicity to neurons. Co-application of SOD (100 U/ml) and SIN-1 (500 μM) caused significant neurotoxicity. The neuronal survival was 11.6±1.2% of control. **p<0.01 was obtained when the SIN-1 plus SOD group was compared to the control group. A representative experiment of three that were performed is shown.

FIG. 8B is a time course graph demonstrating that the interaction of SIN-1 with SOD caused time-dependent H₂O₂ formation. SIN-1 was incubated with SOD or PQQ in the absence or presence of catalase for selected times and then the H₂O₂ concentration was assayed. In each condition, the background, represented by the absorbance obtained in the presence of catalase (100 U/ml), was subtracted. Four hours after the reaction of SIN-1 with SOD or PQQ, the H₂O₂ concentrations were 85.2±0.2 μM and 27±0.8 μM, respectively. A representative experiment of three that were performed is shown.

FIG. 9 is a series of histograms demonstrating that the potentiation NMDA receptor agonists and nitric oxide donors have synergistic effects in producing neurotoxicity.

FIG. 9A is a histogram that shows that in neurons exposed to NMDA (N; 20 μM), DETA/NO (D; 300 μM), and NMDA plus DETA/NO (N+D) for one hour, and 23 hr later, the viability of neurons was assayed by examining the reduction of Alamar Blue by living cells. ***: p<0.001 compared to other groups.

FIG. 9B is a histogram that shows that in neurons exposed to kainate (K; 20 μM), DETA/NO (D; 300 μM), and DETA/NO plus kainate (K+D) for 1 hr, the toxicity was examined at 24 hr. ***: p<0.001 compared to other groups.

FIG. 10 is a series of histograms demonstrating that PQQ and free radical spin traps blocked neurotoxicity induced by four hour exposure of NMDA plus DETA/NO to neurons.

FIG. 10A is a histogram showing that PQQ blocks neurotoxicity induced by a four hour exposure of NMDA plus DETA/NO. ‘N+D’ represents NMDA plus DETA/NO. “***”: p<0.001, comparing the N+D group to the PQQ treated group. A representative experiment of four that were performed is shown.

FIG. 10B is a histogram that shows that free radical spin traps block neurotoxicity induced by a four hour exposure of NMDA plus DETA/NO. Free radical spin traps (PBN at 10 mM and TEMPO at 1 mM) were applied 1 hr before neurons were exposed to NMDA (20 μM) plus DETA/NO (300 μM). Four hours later, the neurotoxicity was examined. “*”: p<0.05, comparing the NMDA plus DETA (N+D) only group (control) to the N+D drug treated groups. A representative experiment of four that were performed is shown.

FIG. 11 is a series of histograms demonstrating that PQQ, but not free radical spin traps, blocks neurotoxicity induced by 24 hr exposure of NMDA plus DETA/NO to neurons.

FIG. 11A demonstrates the ability of PQQ to block neurotoxicity induced by 24 hr exposure of NMDA plus DETA/NO. ‘N+D’ represents NMDA plus DETA/NO. “***”: p<0.001, comparing the N+D group to the PQQ treated group. A representative experiment of four that were performed is shown.

FIG. 11B is a histogram that shows how free radical spin traps have no demonstrable effect on neurotoxicity induced by 24 hr exposure of NMDA plus DETA/NO. Free radical spin traps (PBN at 10 mM and TEMPO at 1 mM) were applied 1 hr before neurons were exposed to NMDA (20 μM) plus DETA/NO (300 μM). Twenty-four hours later, the neurotoxicity was examined. A representative experiment of four that were performed is shown.

FIG. 12 is a dose-response curve demonstrating that PQQ is protective against SIN-1 induced cell toxicity in oligodendrocytes. Oligodendrocytes were exposed to various concentrations of SIN-1 (0, 0.25, 0.5, 1 and 2 mM) in EBSS for 2 hr with or without PQQ (100 μM), then washed with HBSS containing 0.1% BSA for 2 times. Oligodendrocytes were then returned to the culture medium. The neurotoxicity was examined at 24 hr. The EC₅₀ of SIN-1 toxicity in oligodendrocytes was 345±91 μM. PQQ at 100 μM completely blocked death of oligodendrocytes induced by SIN-1 at various concentrations.

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

Definitions

For convenience, certain terms used in the specification, examples, and appended claims are collected here. Unless otherwise defined, all technical and scientific terms used herein have the same meaning as commonly understood by one of ordinary skill in the art to which this invention pertains. However, to the extent that these definitions vary from meanings circulating within the art, the definitions below are to control.

“Neurotoxic” and “neurotoxicity” include any compound or event that is cytostatic or cytotoxic to neural cells.

“Neurological damage” includes any chronic or acute pathological injury involving the nervous system and/or associated tissue, including stroke, ALS, spinal cord injury, and white matter injury (e.g., such as injury associated with cerebral palsy or multiple sclerosis).

“White matter” includes any usually whitish nervous tissue of the CNS consisting of neurons and their myelin sheaths, also known as the substantia alba. White matter injury is prevalent in acute injury, such as perinatal white matter injury (“Shaken Baby Syndrome”), and in chronic diseases including multiple sclerosis and cerebral palsy. Oligodendrocytes are critical targets in an effort to prevent or reverse white matter injury or damage.

“Neurological disorder” includes any disease or disorder characterized by a neurological symptom or manifestation, including Alzheimer's disease, Parkinson's disease, epilepsy, schizophrenia, dementia, Huntington's Disease, cerebral palsy, and multiple sclerosis.

“Peroxynitrite-induced neurotoxicity” includes any cytostatic or cytotoxic event induced or accelerated by a direct or indirect action of peroxynitrite, including nitration or oxidation of biological compounds such as amino acids, polypeptides, lipids, nucleic acids, hormones, and signaling molecules (e.g., gamma-amino butyric acid, gamma-hydroxybutyric acid, dopamine, seratonin, acetylcholine, and glutamate). In normal mammalian tissues, peroxynitrite is generally present at non-toxic levels. Increases with peroxynitrite levels are associated with pathological conditions, diseases and disorders, such as neurotoxicity.

“Peroxynitrite-induced cell death” includes any cell death event induced or accelerated by a direct or indirect action of peroxynitrite, e.g., by apoptosis, necrosis, or other cellular mechanisms.

“Subject” includes living organisms such as humans, monkeys, cows, sheep, horses, pigs, cattle, goats, dogs, cats, mice, rats, cultured cells therefrom, and transgenic species thereof. In a preferred embodiment, the subject is a human. Administration of the compositions of the present invention to a subject to be treated can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced as indicated by the exigencies of the therapeutic situation.

“Substantially pure” includes compounds, e.g., drugs, proteins or polypeptides that have been separated from components that naturally accompany it. Typically, a compound is substantially pure when at least 10%, at least 20%, at least 50%, at least 60%, at least 75%, at least 90%, or at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state. Included within the meaning of the term “substantially pure” are compounds, such as proteins or polypeptides, which are homogeneously pure, for example, where at least 95% of the total protein (by volume, by wet or dry weight, or by mole percent or mole fraction) in a sample is the protein or polypeptide of interest.

“Administering” includes routes of administration which allow the compositions of the invention to perform their intended function, e.g., treating or preventing neural injury caused by peroxynitrite. A variety of routes of administration are possible including, but not necessarily limited to, parenteral (e.g., intravenous, intraarterial, intramuscular, subcutaneous injection), oral (e.g., dietary), topical, nasal, rectal, or via slow releasing microcarriers depending on the disease or condition to be treated. Oral, parenteral and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980)).

“Effective amount” includes those amounts of pyrroloquinoline quinone which allow it to perform its intended function, e.g., treating or preventing, partially or totally, neurological injury caused by peroxynitrite as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg/day to about 500 mg/kg/day, advantageously from about 0.1 mg/kg/day to about 100 mg/kg/day. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Specific binding” or “specifically binds” includes proteins, such as an antibody which recognizes and binds an pyrroloquinoline quinone or a ligand thereof, but does not substantially recognize or bind other molecules in a sample.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, e.g., in Remington's Pharmaceutical Sciences.

“Unit dose” includes a discrete amount of the pharmaceutical composition comprising a predetermined amount of the active ingredient.

Combination therapy” (or “co-therapy”) includes the administration of a pyrroloquinoline quinone compound and an antioxidant as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Excessive or inappropriate generation of nitric oxide has been thought to be one of the major causes of a variety of acute neurological disorders, as well as chronic neurodegenerative diseases. In the central nervous system, nitric oxide is produced by neuronal nitric oxide synthase (nNOS), as well as by inducible NOS (iNOS) located in astrocytes and microglia. Nitric oxide is generally considered to be not toxic itself, but to react with superoxide to form peroxynitrite, a potent and toxic oxidant.

Nitric oxide has been implicated in NMDA receptor mediated neurotoxicity. In nNOS knockout mice, the area of infarction produced in a focal ischemia model was significantly less than in control animals of similar genetic background but with a normal nNOS gene, indicating that nitric oxide may play an important role in stroke. Later studies suggested that the neurotoxicity of nitric oxide in ischemic stroke depends upon its conversion to peroxynitrite.

Pyrroloquinoline quinone (PQQ) is a water soluble anionic quinone that can transfer electrons catalytically between a variety of reductants and oxidants, and may be part of a soluble electron transport system in eukaryotic cells. PQQ proper is of the general structure

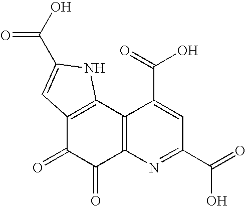

“Pyrroloquinoline quinone” includes any member of the pyrroloquinoline quinone family having chemical similarity, including closely related isomeric and stereoisomeric analogs of PQQ (See e.g., Zhang et al., 1995, Biochem. Biophys. Res. Commun. 212: 41-47, 1995). PQQ is also known as methoxatin. PQQ is found in animal tissues and fluids. Without wishing to be bound by theory, PQQ may act in part as a free-radical scavenger, particularly of reactive nitrogen species (RNS) and reactive oxygen species (ROS). There is strong evidence for involvement of RNS in Parkinson's disease, ALS, Huntington's disease and Alzheimer's disease.

Consistent with its redox cycling potential, in the presence of reductants, PQQ may initiate superoxide production, while in the setting of oxidative stress, it can act as an antioxidant to prevent cell injury. PQQ is able to oxidize the redox modulatory site of NMDA receptors, thus conferring protection against NMDA or glutamate mediated cell injury in cultured neurons. Although PQQ has been shown to be effective in an animal model of focal ischemia and epilepsy, the protective mechanism is far from understood. Without limitation to any particular theory or mechanism of action, the protective effect of PQQ is due, at least in part, to an effect on the synthesis or clearance of RNS. The protective effect of PQQ is also due, at least in part, to the ability of PQQ to reduce endogenous levels of peroxynitrite.

Compositions containing substantially purified pyrroloquinoline quinone may include pyrroloquinoline quinone alone, or in combination with other components such as amino acids, antioxidants (e.g., vitamins A, C and E; selenium), glutamine receptor antagonists or inverse agonists, agents that promote mitochondrial function (e.g., creatine, coenzyme Q) and metal chelators (e.g., zinc). Pyrroloquinoline quinone may be substantially purified by any of the methods well known to those skilled in the art. (See, e.g., E. J. Corey and Alfonso Tramontano, J. Am. Chem. Soc., 103, 5599-5600 (1981); J. A. Duine, Review Ann. Rev. Biochem. 58, 403 (1989)). In an embodiment of the invention, compositions contain pyrroloquinoline quinone and one or more amino acids (e.g., glycine), such that imidazole products (e.g., imidazolopyrroloquinoline) are formed. Imidazole-PQQ derivatives or amino acid-PQQ combinations are believed to allow higher tolerable doses of PQQ.

“Antioxidants” include those commonly referred to in the art, and, e.g., substances that interact with and/or stabilize free radicals, such as vitamins E, C, carotenoids, and β-carotene. Other compounds which may be classified as antioxidants include free radical spin traps. Spin traps are species that react with reactive free radicals to produce fairly stable, unreactive free radicals, thus blocking the free radicals from damaging cellular components. Examples of spin traps include nitrones such as alpha-phenyl-N-tert butyl nitrone (PBN); 5,5-dimethyl-1-pyrroline-N-oxide (DMPO); 1-diethyoxyphosphoryl-1-methyl pyroline-N-oxide (DEPMPO), alpha-(4-pyridyl-1-oxide)-N-tert-butyl nitrone (POBN), and 2,2,6,6-tetramethyl-1-piperidinyl-1-oxyl (TEMPO).

It has surprisingly been found that not only are PQQ compounds more effective than antioxidants such as vitamin E in preventing or reducing peroxynitrite-induced neurotoxicity, but that combinations of PQQ compounds with antioxidants or spin traps are expected to be particularly effective as a combination therapy.

The pyrroloquinoline quinone of the invention is, in one embodiment, a component of a pharmaceutical composition, which may also comprise buffers, salts, other proteins, and other ingredients acceptable as a pharmaceutical composition. The invention also includes a modified form of pyrroloquinoline quinone, which is capable of preventing or reducing peroxynitrite-induced neurotoxicity as described herein.

The structure of the therapeutic compounds of this invention may include asymmetric carbon atoms. It is to be understood accordingly that the isomers (e.g., enantiomers and diastereomers) arising from such asymmetry are included within the scope of this invention. Such isomers can be obtained in substantially pure form by classical separation techniques and by sterically controlled synthesis. For the purposes of this application, unless expressly noted to the contrary, a therapeutic compound shall be construed to include both the R or S stereoisomers at each chiral center. In certain embodiments, an therapeutic compound of the invention comprises a cation. If the cationic group is hydrogen, H⁺, then the therapeutic compound is considered an acid. If hydrogen is replaced by a metal ion or its equivalent, the therapeutic compound is a salt of the acid. Pharmaceutically acceptable salts of the therapeutic compound are within the scope of the invention, e.g., pharmaceutically acceptable alkali metal (e.g., Li⁺, Na⁺, or K⁺) salts, ammonium cation salts, alkaline earth cation salts (e.g., Ca²⁺, Ba²⁺, Mg²⁺), higher valency cation salts, or polycationic counter ion salts (e.g., a polyammonium cation). (See, e.g., Berge et al. (1977) “Pharmaceutical Salts”, J. Pharm. Sci. 66:1-19). It will be appreciated that the stoichiometry of an anionic compound to a salt-forming counter ion (if any) will vary depending on the charge of the anionic portion of the compound (if any) and the charge of the counter ion. Preferred pharmaceutically acceptable salts include a sodium, potassium or calcium salt, but other salts are also contemplated within their pharmaceutically acceptable range.

The invention relates to methods of treating or preventing peroxynitrite-induced neurotoxicity, such as occurs during an acute or chronic neurological disorder, in a subject. This is done by administering to the subject an effective amount of a pyrroloquinoline quinone compound, wherein peroxynitrite is not detectable at toxic levels in the mammalian subject, such that peroxynitrite-induced neurotoxicity is prevented or inhibited.

Neuroprotective signaling pathways are known in the art. These pathways may be targeted for enhancement in patients in need of neuroprotection, by administering pyrroloquinoline quinone in an amount effective to enhance or maintain the effect of neuroprotective signaling pathway while inhibiting peroxynitrite formation.

Nitric oxide is itself generally non-toxic, but reacts with superoxide to form the potent toxicant, peroxynitrite. As such, administration of PQQ, administered in vivo is an effective treatment for inhibiting or preventing damage to neural cells by peroxynitrite. Administration of the compounds of the invention may be done where clinically necessary or desirable prior to the formation of peroxynitrite, e.g., at the onset of reperfusion, or prior to reperfusion.

The invention also encompasses methods of inducing cell toxicity in neural tissue by contacting the tissue with an effective amount of a pyrroloquinoline quinone compound that is capable of interacting with peroxynitrite present in the tissue, such that neural cell death increases. Induction of cell death in tissue such as neural tissue, e.g., neural cell death, is advantageous in situations characterized by aberrant cell proliferation, differentiation, or de-differentiation, e.g., cancers such as neuroblastoma, or other conditions involving malignancy wherein peroxynitrite is formed in pathological levels.

The invention encompasses methods of treating or preventing neurological injury caused by peroxynitrite in a subject, where PQQ is administered to a subject in need thereof, such that neurological injury is prevented or decreased. In certain embodiments, the PQQ is administered at a concentration of less than about 10 μM. In other embodiments, the PQQ is administered at a concentration in the range of about 10 nM to about 500 μM, about 100 nM to about 500 μM, 10 μM to about 100 μM, and 100 μM to about 500 μM. In still other embodiments of the invention, the PQQ is administered at a concentration such that the concentration of PQQ at the site of neural tissue is in the range of 10 nM to about 500 μM. PQQ may also be administered as a function of the subject's body weight. In some embodiments of the invention, PQQ is administered at a concentration of between about 1 μg/kg to 1 g/kg of a subject's body weight, including less than 500 mg/kg, 250 mg/kg, 100 mg/kg, 10 mg/kg, 5 mg/kg, 2 mg/kg, 1 mg/kg, 500 μg/kg, 250 μg/kg, 100 μg/kg, 10 μg/kg, 5 μg/kg, 2 μg/kg or 1 μg/kg. In further embodiments of the invention, the PQQ is administered at a non-toxic concentration, which includes concentrations of PQQ which are cytostatic but not cytotoxic, and concentrations which are cytotoxic to cell types other than the intended one or more cell types (e.g., neural cells). The determination of the cytotoxicity of a known concentration of PQQ to one or more cell types is within the abilities of one of ordinary skill in the art.

The neurological injury that can be treated or prevented by the methods and compositions of the present invention includes all neurological injury caused or affected by peroxynitrite. Such injury includes, but is not limited to, hypoxia, anoxia, carbon monoxide poisoning, lead poisoning, ischemia, CNS trauma, hypoglycemia, siezures, stroke, ALS, alchoholism-related conditions, AIDS dementia complex, Parkinson's Disease, Alzheimer's Disease, neurolathyrism, hepatic encephalopathy, Tourette's syndrome, mitochondrial abnormalities (e.g., MELAS syndrome, Rett syndrome, homocysteinurea, hyperprolinemia, hyperglycinemia, hydroxybutyric aminoacidurea and sulfite oxidase deficiency), and Guam disease. All or a portion of the central nervous system or peripheral nervous system may be injured, including associated blood vessels and/or tissue.

The invention also encompasses methods for preventing, inhibiting or reducing neurological damage incident to a stroke in a mammalian subject at risk thereof by administering to the subject an effective amount of a pyrroloquinoline quinone compound prior to a measurable increase in peroxynitrite formation in the mammalian subject, such that neurological damage incident to stroke is prevented, inhibited or reduced.

The invention further encompasses methods for treating a chronic neurological disorder in a mammalian subject in need thereof by administering to the subject an effective amount of a pyrroloquinoline quinone compound at a point wherein peroxynitrite is generally undetectable at toxic levels, such that the chronic neurological disorder is reduced or eliminated.

The invention encompasses methods for preventing or inhibiting peroxynitrite-induced cell death in a mammalian subject at risk of or undergoing an acute traumatic neurological event by administering to the subject an effective amount of a pyrroloquinoline quinone compound at such time wherein peroxynitrite is generally detected at physiological, non-toxic levels, such that peroxynitrite-induced cell death is prevented or inhibited. The acute traumatic neurological event can include surgery, radiation treatment, infection, organ or tissue transplant, trauma, carbon monoxide poisoning, cardiac arrest, and birth asphyxiation.

The invention encompasses the preparation and use of pharmaceutical compositions comprising a compound useful for the prevention or reduction of peroxynitrite-induced neural damage as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a pharmaceutically acceptable ester or salt, such as in combination with a physiologically acceptable cation or anion, as is well known in the art. Further, the pyrroloquinoline quinone may contain pharmacologically acceptable additives (e.g., carrier, excipient and diluent), stabilizers or components necessary for formulating preparations, which are generally used for pharmaceutical products, as long as it does not adversely affect the efficacy of the preparation, e.g., in decreasing or inhibiting peroxynitrite-induced neural injury. In some embodiments, the PQQ is formulated in polyethylene glycol or liposomes to enhance central nervous system absorption and efficacy.

Examples of additives and stabilizers include saccharides such as monosaccharides (e.g., glucose and fructose), disaccharides (e.g., sucrose, lactose and maltose) and sugar alcohols (e.g., mannitol and sorbitol); organic acids such as citric acid, maleic acid and tartaric acid and salts thereof (e.g., sodium salt, potassium salt and calcium salt); amino acids such as glycine, aspartic acid and glutamic acid and salts thereof (e.g., sodium, calcium or potassium salt); surfactants such as polyethylene glycol, polyoxyethylene-polyoxypropylene copolymer and polyoxyethylenesorbitan fatty acid ester; heparin; and albumin.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. The preferred mode is intravenous administration.

The pyrroloquinoline quinone and the above-mentioned ingredients are admixed as appropriate to give powder, granule, tablet, capsule, syrup, injection and the like. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses. The amount of the active ingredient is generally equal to the dosage of the active ingredient, which would be administered to a subject, or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Particularly contemplated additional agents include anti-emetics and scavengers such as cyanide and cyanate scavengers. Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include potato starch and sodium starch glycollate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in, e.g., U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, a gel or cream or solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Additional delivery methods for administration of compounds include a drug delivery device, such as that described in U.S. Pat. No. 5,928,195.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or diglycerides. Other parentally administrable formulations that are useful include those, which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface-active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e., by rapid inhalation through the nasal passage from a container of the powder held close to the nose.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically administrable formulations that are useful include those, which comprise the active ingredient in microcrystalline form or in a liposomal preparation.

The mixture of pyrroloquinoline quinone and pharmacologically acceptable additives is preferably prepared as a lyophilized product, and dissolved when in use. Such preparation can be prepared into a solution containing about 0.01-100.0 mg/ml of pyrroloquinoline quinone, by dissolving same in distilled water for injection or sterile purified water. More preferably, it is adjusted to have a physiologically isotonic salt concentration and a physiologically desirable pH value (pH 6-8).

While the dose is appropriately determined depending on symptom, body weight, sex, animal species and the like, it is generally assumed that treatment options holding the blood concentration at about 1 μM will be preferred. This plasma concentration may be achieved through administration of one to several doses a day. When pyrroloquinoline quinone is to be administered to a subject, 0.1 ng to 10 mg/kg body weight (e.g., 1 ng to 1 mg/kg body weight) of pyrroloquinoline quinone can be given intravenously.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLES

These Examples are provided for the purpose of illustration only and the invention should in no way be construed as being limited to these Examples, but rather should be construed to encompass any and all variations which become evident as a result of the teaching provided herein.

The following experiments were conducted to further describe the ability of early administration of pyrroloquinoline quinone to prevent peroxynitrite-induced neural damage.

### Example 1

### Pyrroloquinoline Quinone Suppression of Peroxynitrite Formation and Action

General Methods and Materials: Chemicals and Reagents.

Nitric oxide donors, SIN-1 and peroxynitrite were obtained from Cayman Chemical Co. (Ann Arbor, Mich.). Purified bovine hemoglobin was obtained from Biopure Corporation (Cambridge, Mass.). Tissue culture media were obtained from Life Technologies (Grand Island, N.Y.) unless noted otherwise. Mn (III) tetrakis (4-benzoic acid) porphyrin chloride (MnTBAP) was obtained from Calbiochem (San Diego, Calif.). PQQ was obtained from Fluka (Buchs, Switzerland). All other reagents were from Sigma (St. Louis, Mo.). Mouse anti-nitrotyrosine monoclonal antibody was from Upstate Biotechnology (catalog #05-233; Lake Placid, N.Y.). Horseradish peroxidase-conjugated sheep anti-mouse immunoglobulin G (IgG) was from Amersham Pharmacia Biotech (Piscataway, N.J.). Enhanced chemiluminescence (ECL) was used for detection using a kit from NEN Life Science (Boston, Mass.)

Tissue Culture.

Neuronal cultures were prepared from embryonic day 16 Sprague Dawley rat fetuses using methods similar to those previously described (Dichter, 1978) but modified to produce cultures that contained <1% astrocytes (Rosenberg and Aizenman, 1989; Rosenberg, 1991; Rosenberg et al., 1992; Wang et al., 1998). Cultures were initially plated on poly-L-lysine-coated 24-well plastic plates (Costar, Cambridge, Mass.) using an 80:10:10 (v/v) mixture ofDMEM (Life Technologies 11960-010), Ham's F-12 (Sigma, N-4888), heat-inactivated iron-supplemented calf serum (Hyclone A2151), containing 2 mM glutamine, 25 mM HEPES, 24 U/ml penicillin, and 24 μg/ml streptomycin and maintained in a 5% CO₂ (balance air) incubator at 36° C. Cell proliferation was inhibited by exposure to 5 μM cytosine arabinoside at 24 hr in vitro for 72 hr. On the fourth day of culture, the medium was completely removed and replaced with 90% MEM, 10% Nuserum IV (Collaborative Research), 2 mM glutamine, 5 mM HEPES, containing 50 units/ml superoxide dismutase (Boehringer Mannheim, Indianapolis, Ind.), 20 units/ml catalase (Sigma CV-40), total glucose 11 mM, and total sodium bicarbonate 9.3 mM, plus 2% B27 supplement (Life Technologies 17504-036). Medium was not subsequently changed. To prevent evaporation of water, culture dishes were kept in wet dishes containing filter paper that was always kept wet. Protein was approximately 100 μg/well (Rosenberg et al., 2000) determined with a bovine serum albumin standard using bicinchoninic acid (Smith et al., 1985).

Application of Reagents.

In all experiments, 24-well plates were first washed three times in Hank's balanced salt solution (HBSS) containing 0.1% bovine serum albumin (BSA), and then they were placed in Earle's balanced salt solution (EBSS, which is composed of 116 mM NaCl, 5.4 mM KCl, 1.8 mM CaCl₂, 0.8 mM MgSO₄, 26 mM NaHCO₃, 1 mM NaH₂PO₄ and 5.5 mM D-glucose). Experiments were carried out exclusively in EBSS. MK-801, hemoglobin, bovine superoxide dismutase, catalase and MnTBAP were applied 15 minutes before the cells were exposed to nitric oxide donors, 3-morpholinosydnonimine (SIN-1), and peroxynitrite. PQQ was directly dissolved in EBSS.

Survival Assay.

Survival of cells was determined after 24 hr exposure to the test compounds by assessing the ability of cultures to reduce Alamar Blue (Trek Diagnostic Systems, Inc., Westlake, Ohio) as an index of viability (McGahon et al., 1995). Details of this procedure have been provided elsewhere (Back et al., 1998, 1999). In brief, the test medium was removed after 24 hr, and then replaced with EBSS plus a 1:100 dilution of Alamar Blue. After 2 hr exposure, the medium was diluted 50-fold with water, and the fluorescence intensity was determined in a Hitachi fluorescence spectrophotometer using an excitation wavelength of 530 nm and an emission wavelength of 590 nm. The photomultiplier voltage was set at 700 V. In some experiments, the cell viability determined by Alamar Blue was compared to that determined by the assay of lactate dehydrogenase (LDH) release (Koh and Choi, 1987).

Assay of Intracellular ATP.

The Bioluminescent Somatic Cell Assay kit from Sigma (catalog No. FL-ASC) was used for assay of ATP as previously described (Rosenberg et al., 2000). At selected time points after addition of drug or vehicle, medium was removed, and then replaced with 200 μl of cell lysis agent. After brief agitation, the medium was transferred to microcentrifuge tubes that contained 200 μl of water. The tubes were placed on wet ice until assay. ATP assay mix was used at 25-fold dilution, and 100 μl of the extract was assayed.

Western Blot Analysis.

BSA (0.5 mg/ml) in EBSS with or without PQQ was exposed to SIN-1 or peroxynitrite for 1 hr, and then 4× sample buffer (40% glycerol, 20% β-mercaptoethanol, 0.004% bromophenol blue, 6.4% SDS, 25% 1M Tris-HCl, pH 6.8) was added. After the samples were heated for 5 min, 2 μg protein per lane was subjected to SDS-PAGE analysis, followed by transfer to PVDF membrane (NEN Life Science, Boston, Mass.) and reacted with a mouse anti-nitrotyrosine monoclonal antibody (Upstate Biotechnology). Proteins were visualized with horseradish peroxidase-conjugated sheep anti-mouse immunoglobulin G (IgG) followed by detection with enhanced chemiluminescence.

Measurement of Intracellular cGMP.

Intracellular cGMP was assayed by enzyme-linked immunoassay (Amersham). Cultures were washed three times, and then medium was replaced by EBSS with or without PQQ or SOD. The non-specific phosphodiesterase inhibitor, 3-isobutyl-1-methylxanthine (IBMX) at 100 μM was present in all conditions. DEA/NO, SIN-1 and peroxynitrite were added for 5 min, and then the medium was replaced with 200 μl of releasing reagent provided in the kit. The plates were agitated for 20 min at room temperature, and 20 μl of acetylation reagent was added. After 5 min, 50 μl samples were taken for assay.

Assay of H₂O₂ Produced by the Reaction of SIN-1 with SOD or PQQ

The method described by Yakes and Van Houten (1997) was used, exploiting the oxidation of o-diansidine dihydrochloride (o-DD, Sigma D-3252) in the presence of horseradish peroxidase (Sigma P-8250). Briefly, SIN-1 (1 mM) was incubated with SOD (100 units/ml) or PQQ (100 μM) in the absence or presence of catalase (100 units/ml) in EBSS for various times. Then o-DD was added to a final concentration of 250 μM and 2.5 units/ml horseradish peroxidase were added. After 1 hr incubation at 37° C., the absorbance at 470 nm was measured. The concentration of H₂O₂ in samples was calculated based on a standard curve obtained by the reaction of various concentrations of H₂O₂ with o-DD and horseradish peroxidase.

Statistics

Statistical significance was assessed using ANOVA with the Tukey-Kramer post-hoc multiple comparison test. Statistical analysis was performed using the Instat program from GraphPad Software (San Diego, Calif.). Representative experiments are shown unless noted otherwise. Experiments were performed with triplicate samples, and the data are expressed as mean SD or SEM, as is appropriate. All experiments were repeated at least three times.

SIN-1 Toxicity to Neurons is Mediated by the Formation of Peroxynitrite

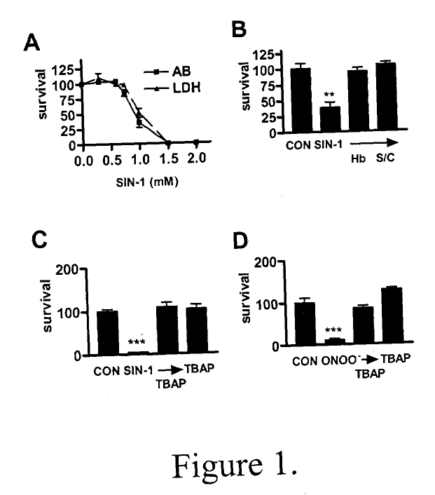

In the presence of oxygen, SIN-1 generates nitric oxide (NO), as well as superoxide (O₂⁻) (Hogg et al., 1992). NO interacts with O₂⁻ in a diffusion-limited reaction resulting in the formation of peroxynitrite (ONOO⁻) (Beckman et al., 1990). In cultured rat forebrain neurons, SIN-1 caused neuronal death in a concentration-dependent manner (FIG. 1). In five experiments, we compared LDH release (Koh and Choi, 1987) and Alamar Blue reduction (Back et al., 1998, 1999) methods for assay cell survival and found that they produced similar results (FIG. 1A). The toxicity caused by 1 mM SIN-1 was completely blocked by a NO □scavenger, hemoglobin (Hb, 43 μM), as well as superoxide dismutase (SOD, 100 U/ml) plus catalase (CAT, 100 U/ml) (FIG. 1B; 3 separate experiments). This result is in agreement with a previous report (Lipton et al., 1993), suggesting that neither nitric oxide itself nor superoxide alone generated by SIN-1 was sufficient to cause cell death. The toxic species was likely to be the adduct formed by reaction of nitric oxide and superoxide, peroxynitrite. MnTBAP, which is a membrane permeable SOD mimic and a scavenger of peroxynitrite but not nitric oxide (Szabo et al., 1996), completely protected against SIN-1 toxicity at 100 μM (FIG. 1C; three separate experiments), consistent with the hypothesis that peroxynitrite is the toxic species. MnTBAP also blocked the neurotoxicity induced by 100 μM peroxynitrite (FIG. 1D; three separate experiments).

Effects of PQQ to Neurotoxicity Induced by SIN-1 or Peroxynitrite

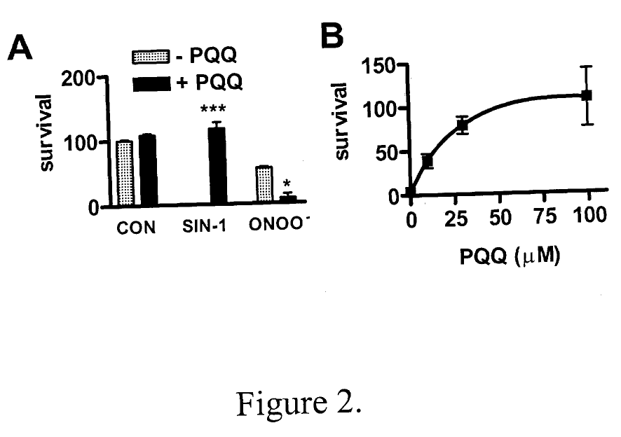

In order to examine whether PQQ interacts with peroxynitrite, the effect of PQQ on SIN-1 and on peroxynitrite-induced neurotoxicity was studied (FIG. 2). PQQ (100 μM) completely protected neurons against SIN-1 (1 mM) induced toxicity (FIG. 2A; three separate experiments). Surprisingly, the neurotoxicity caused by peroxynitrite (100 μM) was not blocked, but was actually potentiated. Without PQQ, the neuronal survival in the presence of peroxynitrite was 49±7.3%, while in the presence of PQQ, only 11±6% of neurons survived. This result suggested that the protective effect of PQQ against SIN-1 toxicity might be due to preventing peroxynitrite formation by eliminating superoxide rather than by scavenging peroxynitrite. This might occur if PQQ were a scavenger of nitric oxide or of superoxide. FIG. 2B shows the concentration-dependence of the protective effect of PQQ against SIN-1 toxicity. In three experiments that were performed, the EC₅₀ value was 15±8.4 μM.

PQQ Blocks ATP Depletion Induced by SIN-1, But Not by Peroxynitrite

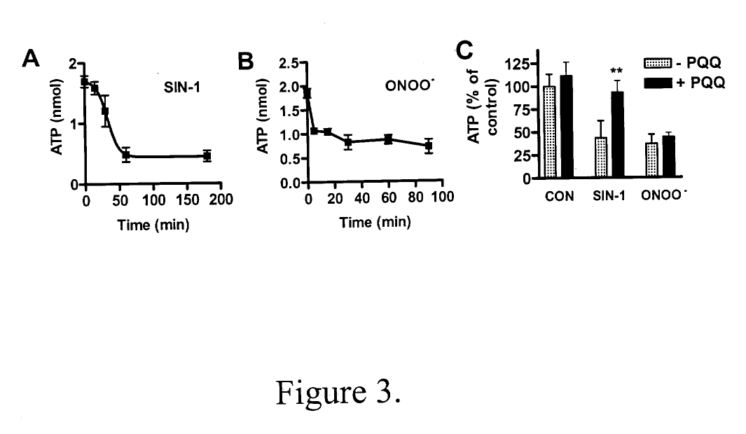

One of the important effects of nitric oxide on cells is mitochondrial dysfunction and energy failure (Pang and Geddes, 1997; Schulz et al., 1997; Heales et al., 1999; Torreilles et al., 1999). In central neurons, both nitric oxide and peroxynitrite produce ATP depletion and mitochondrial membrane depolarization (Brorson et al., 1999). Nitric oxide can also damage DNA, leading to the activation of poly (ADP-ribose) polymerase, depletion of NAD⁺ and consumption of ATP (Zhang et al., 1994). We examined the time dependence of ATP depletion induced by SIN-1 and peroxynitrite in neuronal cultures (FIG. 3). SIN-1 (1 mM) maximally depleted ATP after a 1 hr exposure (FIG. 3A; 3 separate experiments). In contrast, the peak ATP depletion induced by 100 μM peroxynitrite occurred much earlier; after a 5 min exposure, ATP already dropped to 50% of its original levels, and remained relatively constant for the duration of the experiment (FIG. 3B; 3 separate experiments). PQQ (100 μM) significantly blocked ATP depletion caused by a 1 hr exposure to 1 mM SIN-1 (FIG. 3C; 3 separate experiments). ATP concentration increased from 43±15.6% to 84.4±13.9% of control values when PQQ was present. Conversely, PQQ had no effect on 100 μM peroxynitrite induced ATP depletion (FIG. 3C). In the presence of peroxynitrite, ATP levels without or with PQQ were 40.5±11% and 42±5.4% of control values, respectively.

Effect of PQQ on Protein Nitration Caused by SIN-1 or Peroxinitrite

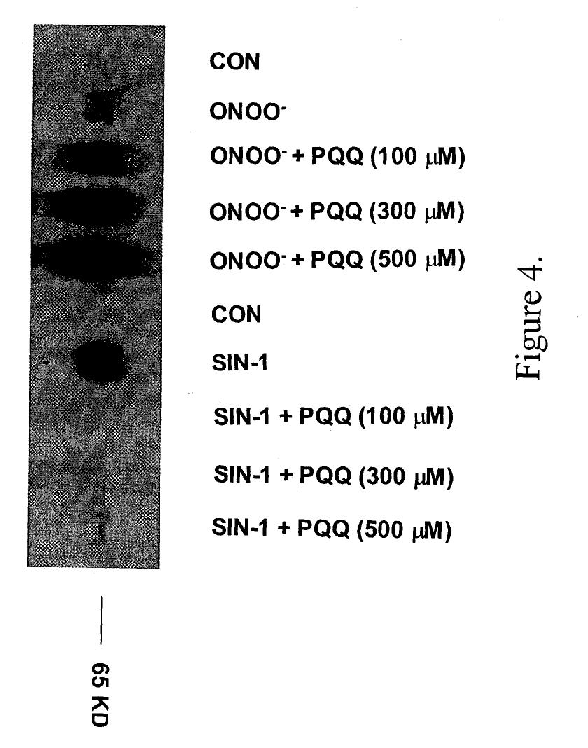

Peroxynitrite is a strong oxidizing and a nitrating agent. Tyrosine nitration in proteins has been used as a specific marker for peroxynitrite mediated cell injury (Ducrocq et al., 1999) and may be the cause of that injury (MacMillan-Crow et al., 1996, 1998). The effect of PQQ on nitration of BSA caused by 1 mM SIN-1 or 100 μM peroxynitrite was examined in a cell-free system by immunoblot using an anti-nitrotyrosine monoclonal antibody. SIN-1 caused strong nitration of BSA, which was completely blocked by PQQ at 100 μM (FIG. 4; 3 separate experiments). In contrast, the nitration caused by peroxynitrite was potentiated by PQQ in a concentration-dependent manner (FIG. 4). This phenomenon was found in three similar experiments.

Nitric Oxide, But Not Peroxynitrite, Caused NMDA Receptor Mediated Cell Injury

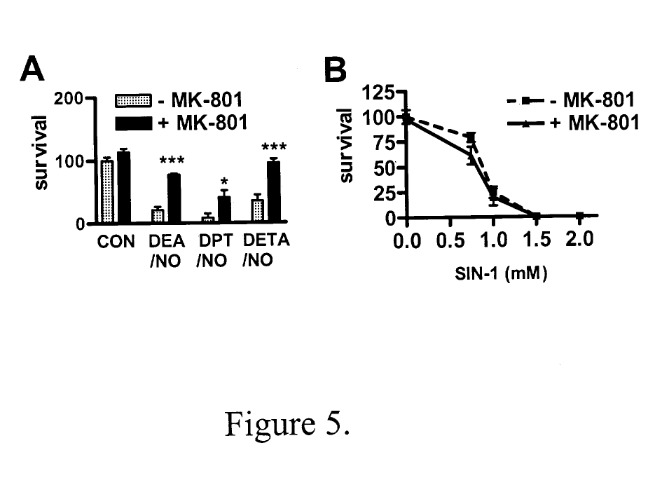

Three nitric oxide donors were used to examine nitric oxide toxicity to neurons in culture: diethylamine NONOate (DEA/NO) with a half-life of 2.1 min (Maragos et al., 1991); dipropylenetriamine NONOate (DPT/NO) with a half-life of 5 hours; and diethylaminetriamine NONOate (DETA/NO) with a half-life of 22 hours (Mooradian et al., 1995). DPT/NO at 1 mM caused complete neuronal death, which could be significantly (p<0.05) blocked by 10 μM MK-801, a non-competitive NMDA receptor antagonist (Wong et al., 1986). The neuronal survival increased from 6.5±5.3% to 37.3±8% (FIG. 5A; 6 separate experiments). A large degree of variability was observed in the neurotoxicity caused by DEA/NO or DETA/NO. In three out of six experiments, 1 mM DEA/NO or DETA/NO did not cause any toxicity. However, in the three out of six experiments in which DEA/NO and DETA/NO were neurotoxic, MK-801 had a clear protective effect. For example, in the experiment shown, using DEA/NO, the neuronal survival increased from 18.2±5.3% to 77.4±3.5% in the MK-801 treated group (FIG. 5A). In the same experiment, using DETA/NO, neuronal survival increased from 27.9±8.0% to 83.1±7.4% in the MK-801 treated group (FIG. 5A). Unlike its protective effect against nitric oxide toxicity, MK-801 at 10 μM did not confer significant protection against the neurotoxicity induced by SIN-1 (FIG. 5B; 3 separate experiments). MK-801 also did not confer protection against peroxynitrite toxicity (data not shown).

PQQ Did Not Block ATP Depletion or Cell Death Induced by Nitric Oxide

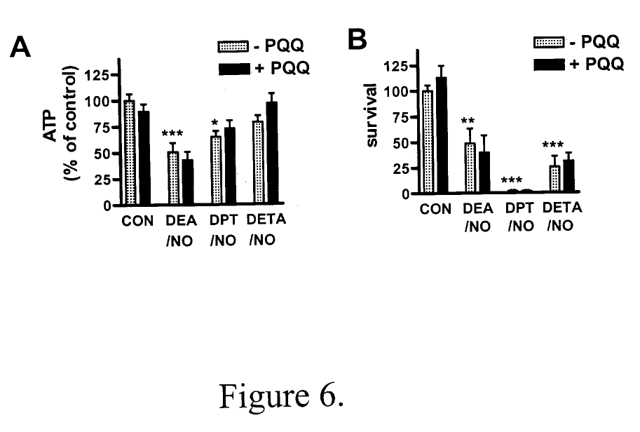

The distinct effects of PQQ on ATP depletion, neuronal death, and protein nitration caused by SIN-1, in contrast to peroxynitrite, support the hypothesis that PQQ may directly scavenge either superoxide or nitric oxide or both. One hour after DEA/NO (1 mM) and DPT/NO (1 mM) exposure, the levels of ATP dropped to 51.6±5.8% and 62.6±4.5% of control values, respectively (FIG. 6A; 3 separate experiments). However, PQQ at 100 μM did not block ATP depletion (FIG. 6A), nor did it prevent cell death caused by any of the three nitric oxide donors 12 tested (FIG. 6B; 3 separate experiments). This suggested that PQQ was unable to directly scavenge nitric oxide.

Effect of PQQ on cGMP Accumulation Induced by Nitric Oxide or Peroxynitrite

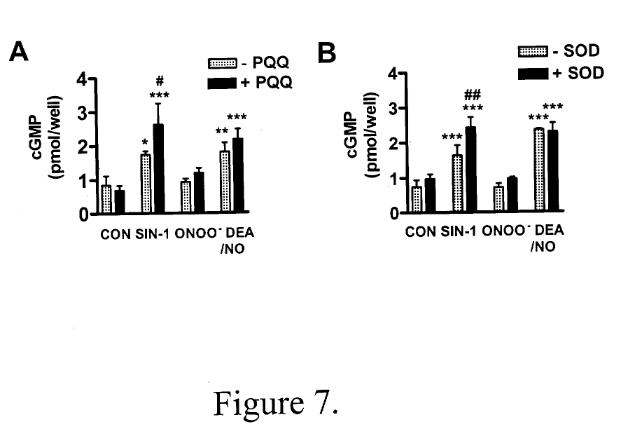

Exogenous, as well as endogenous, nitric oxide generated from nitric oxide synthase (NOS) can activate soluble guanylyl cyclase (sGC), leading to the production of cGMP (Murad et al., 1993). To further examine the interaction between PQQ and nitric oxide, the effect of 100 μM PQQ on cytosolic cGMP accumulation induced by DEA/NO (10 μM), SIN-1 (100 μM), or peroxynitrite (100 μM) was studied (FIG. 7). DEA/NO and SIN-1 increased cGMP levels to 250±15.5% and 192±10.7% of control values, respectively. In contrast, the content of cGMP was not significantly affected by peroxynitrite (95.6±4.3% of control levels) (FIG. 7A; 4 separate experiments). PQQ did not significantly alter the levels of cGMP in the presence of DEA/NO. However, PQQ potentiated the cGMP accumulation induced by SIN-1 (FIG. 7A).

In the presence of PQQ, cGMP content was increased by SIN-1 to 267±19.5% of control levels. This is significantly higher than the increase induced by SIN-1 alone (p<0.05). This result is consistent with formation of peroxynitrite in solutions of SIN-1 reducing the amount of nitric oxide available for interaction with guanylyl cyclase. SOD had an effect similar to that of PQQ (FIG. 7B; 4 separate experiments). These results are consistent with previous reports showing that PQQ is able to scavenge superoxide (Paz et al., 1992; Urakami et al., 1997).

SOD Potentiated SIN-1 Toxicity

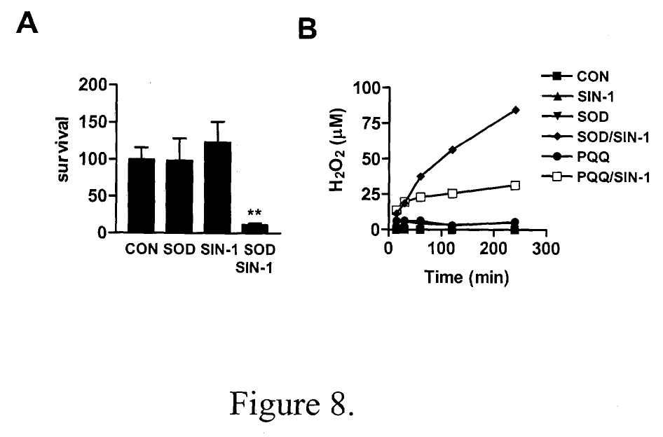

Since PQQ may be acting against SIN-1 toxicity as a superoxide scavenger, we tested the effect of SOD on SIN-1 toxicity. SOD at 100 U/ml or SIN-1 at 500 μM did not have any toxicity to neurons in these experiments (FIG. 8A; 3 separate experiments). However, co-application of SOD plus SIN-1 caused reduction of neuronal survival to 16.8±6.3% of control levels (FIG. 8A; 3 separate experiments). In contrast, without catalase, PQQ completely protected against neurotoxicity induced by SIN-1 (FIG. 2). Two explanations seemed possible: 1) PQQ might scavenge superoxide forming little or no hydrogen peroxide (H₂O₂); 2) PQQ might scavenge H₂O₂, as well as superoxide. In order to distinguish between these hypotheses, we assayed the production of H₂O₂ by SIN-1 in the presence of PQQ or SOD (FIG. 8B; three separate experiments). We found a time-dependent increase in H₂O₂ concentration when SIN-1 was combined with SOD (FIG. 8B). Addition of catalase brought H₂O₂ to basal levels (data not shown). Less H₂O₂ was formed by reaction of SIN-1 with PQQ. Four hours after reaction of SIN-1 with SOD or PQQ, the concentrations of H₂O₂ were 110±12.4 μM and 14.3±4.3 μM. PQQ did not reduce the oxidation of o-diansidine by H₂O₂ (data not shown), indicating that PQQ could not directly interact with H₂O₂. Therefore, it appears that PQQ can scavenge superoxide without forming toxic levels of H₂O₂.

Several lines of evidence demonstrate that PQQ prevents the formation of peroxynitrite without scavenging it directly: 1) PQQ blocked the neurotoxicity induced by the peroxynitrite generator, SIN-1, but not by nitric oxide donors or peroxynitrite itself; 2) ATP depletion produced by SIN-1, but not by nitric oxide donors or peroxynitrite, was blocked by PQQ; 3) PQQ blocked tyrosine nitration caused by SIN-1, but not by peroxynitrite; 4) PQQ potentiated cGMP accumulation caused by SIN-1, indicating that PQQ scavenges superoxide and increases the amount of nitric oxide interacting with soluble guanylyl cyclase.

PQQ Scavenges Superoxide Without Forming Toxic Levels of H₂O₂

The blockade of peroxynitrite formation by PQQ suggested that PQQ might interact either with nitric oxide or with superoxide. Since PQQ did not block neurotoxicity and ATP depletion induced by nitric oxide donors (DEA/NO, DPT/NO and DETA/NO), and did not prevent cGMP accumulation caused by DEA/NO or SIN-1, it is unlikely that a direct interaction occurs between PQQ and nitric oxide. Therefore, it is possible that PQQ functions as a SOD mimetic (Paz et al., 1992). Using ESR in in vitro experiments, Urakami et al (1997) found that the IC₅₀ value of PQQ in scavenging superoxide was 3.8×10⁻⁸ M. However, in the absence of catalase, the effects of SOD and PQQ on the neurotoxicity induced by SIN-1 were quite different. In the absence of catalase, SOD potentiated SIN-1 mediated toxicity, as has been reported previously (Moro et al., 1998; Trackey et al., 2001), while PQQ was neuroprotective. SOD competes with nitric oxide to react with superoxide (Hogg et al., 1992; Lipton et al., 1993), converting superoxide into hydrogen peroxide (H₂O₂), which then may generate highly reactive hydroxyl radicals (OH) via the Fenton reaction (Halliwell and Gutteridge, 1992). We found that H₂O₂ was, in fact, generated when SOD was present in aqueous solutions of SIN-1. In contrast, reaction of SIN-1 with PQQ resulted in much less production of H₂O₂. This is consistent with PQQ acting primarily as a direct scavenger of superoxide rather than as a SOD mimetic. It has been suggested that scavenging superoxide with PQQ is less expensive to the reducing economy of cells than scavenging superoxide with SOD (Paz et al., 1992; Fluckiger et al., 1993; Bishop et al., 1998). Reduced PQQ can donate reducing equivalents to β-nicotinamide adenine dinucleotide phosphate (NADP⁺), forming NADPH. NADPH can then be used to reduce oxidized glutathione (GSSG) back to its reduced form (GSH) by glutathione reductase (Halliwell and Gutteridge, 1990). Thus, PQQ, serving as an anti-oxidant, may increase the concentration of intracellular reduced glutathione. In contrast, SOD dismutates superoxide to form hydrogen peroxide, which is a substrate of glutathione peroxidase (Halliwell and Gutteridge, 1990). Therefore the production of hydrogen peroxide may deplete cells of reduced glutathione.

PQQ Protection Against SIN-1 Toxicity is Not Due to Oxidation of the NMDA Receptor

We found that nitric oxide donors, SIN-1, and peroxynitrite all caused neurotoxicity in nearly pure cultures of rat forebrain neurons. However, the cell death mechanisms appeared to be different. The neurotoxicity caused by nitric oxide donors (DEA/NO, DPT/NO and DETA/NO) was dependent on NMDA receptor activation, presumably secondary to glutamate release (Montague et al., 1994; Meffert et al., 1994, 1996; Leist et al., 1997), while the toxicity caused by SIN-1 or peroxynitrite was primarily NMDA receptor independent. PQQ has been shown to be protective against NMDA receptor mediated neurotoxicity due to its directly oxidizing the redox site of the NMDA receptor (Aizenman et al., 1992; 1994). Therefore we would expect that PQQ might be protective against the neurotoxicity induced by nitric oxide donors, mediated by NMDA receptor activation, but not the neurotoxicity induced by SIN-1, which is independent of NMDA receptors. However, we found that the opposite was true. PQQ blocked the neurotoxicity induced by SIN-1, but not by nitric oxide donors. Thus, the protective action of PQQ cannot be accounted for by its modification of the redox modulatory site of NMDA receptors. One possibility is that NMDA receptors on neurons are maximally oxidized under our culture conditions. In this case, PQQ could not further oxidize the redox sensitive site of the NMDA receptor.

PQQ Potentiates Protein Nitration and Neurotoxicity Caused by Peroxynitrite

PQQ blocked SIN-1 induced nitration of tyrosine residues in BSA, but it actually potentiated the nitration caused by peroxynitrite. This result is parallel to the observation that PQQ blocked SIN-1 mediated cell death, but potentiated neurotoxicity induced by peroxynitrite, suggesting that there may be a causal relationship between the effect of PQQ on protein nitration and its effect on cell injury caused by RNS. At neutral pH, peroxynitrite becomes peroxynitrous acid, which then forms intermediate compounds having the properties of hydroxyl-like and nitrogen dioxide-like species (Ducrocq et al., 1999). Whether free hydroxyl radicals are generated from the decomposition of peroxynitrous acid is controversial (Koppenol et al., 1992; Kooy et al., 1994, 1997; Van der Vliet et al., 1995). However, nitrogen dioxide is thought to be the species that causes tyrosine nitration in proteins (Ducrocq et al., 1999). A possible explanation for the potentiation of protein nitration by PQQ is that PQQ may act as a scavenger of hydroxyl radicals, thus increasing the formation of nitrogen dioxide from peroxynitrous acid via mass action. It has been found that numerous cellular proteins are nitrated by peroxynitrite, for example, neurofilament (Crow et al.,1997), MnSOD (MacMillan-Crow, et al., 1996, 1998), tyrosine hydroxylase (Ara et al., 1998) and cytochrome C (Cassina et al., 2000). Nitration may inactivate protein function or block signal transduction by blocking tyrosine phosphorylation (Kong et al., 1996). PQQ may therefore offer a mixed benefit by blocking nitration and exerting a neuroprotective effect, or potentiating nitration and cell death once peroxynitrite is formed.

Relevance to Brain Ischemic Injury and Neurodegeneration

It is well documented that nitric oxide plays an important role in ischemia-induced cell injury. Specific nNOS and iNOS inhibitors protected neurons against ischemic injury, and mice with targeted gene disruption of nNOS or iNOS were resistant to ischemic damage. In contrast, inhibitors or knockout of the eNOS gene exacerbated the neuronal damage induced by ischemia (Lu et al., 1997; Huang et al., 1996), suggesting a protective effect of nitric oxide in blood vessels. Therefore, the neuroprotective effect of PQQ in vivo (Jensen et.al., 1994) may be due to blocking peroxynitrite formation in the brain, increasing nitric oxide levels by scavenging superoxide in the blood vessels, or directly oxidizing the redox site of the NMDA receptor. Recently PQQ has been suggested to suppress ictal activity by reversing pathological reduction of the redox site on the NMDA receptor without inhibiting physiological NMDA receptor function (Sanchez et al., 2000).

An increasing body of evidence suggests that nitric oxide and peroxynitrite are involved in chronic neurodegenerative diseases, including Parkinson's disease (Gisson et al., 2000), Huntington's disease (Matthews et al., 1998), Alzheimer's disease (Smith et al., 1997) and amyotrophic lateral sclerosis (ALS) (Beckman et al., 1993; Beal et al., 1997; Crow et al., 1997; Estevez et al., 1999; Catania et al., 2001). Recently, it has been shown that widespread nitrotyrosine formation occurred in the brain and spinal cord of G93A-SOD transgenic mice (Cha et al., 2000). The superoxide dismutase and catalase mimetics EUK-8 and EUK-134 reduced protein tyrosine nitration and prolonged survival of these transgenic animals (Jung et al., 2001). We have shown PQQ to protect neurons against the toxicity of peroxynitrite by preventing its formation. Because PQQ is an essential nutrient, at least in mice (Killgore et al., 1989; Steinberg et al., 1994; Stites et al., 2000), there may be a transport system to bring PQQ into cells and across the blood-brain barrier. Therefore, PQQ or its derivatives are of therapeutic benefit in the treatment of neurodegenerative diseases in which the formation of peroxynitrite plays an important pathogenetic role.

### Example 2

### Pyrroloquinoline Quinone Reduces Endogenous Peroxynitrite Levels

Nitric Oxide Has a Synergistic Effect with NMDA or Kainate in Producing Neurotoxicity

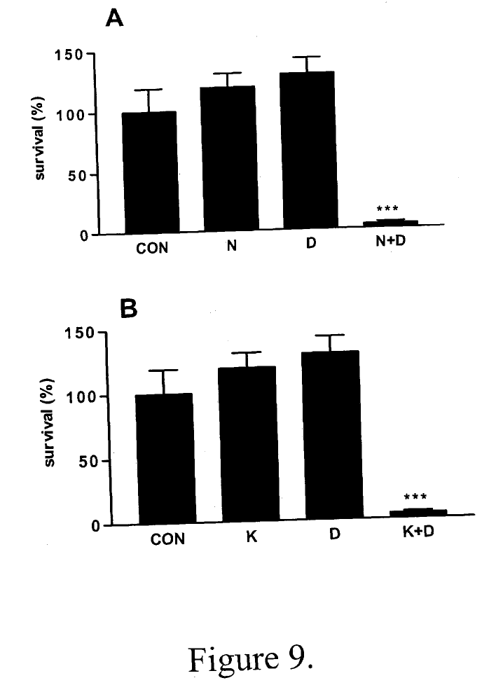

To test the pathophysiological relevance of the protective effect of PQQ against the toxicity of SIN-1, a model system was established in which peroxynitrite formation occurs in a manner closely resembling in vivo conditions. A 24 h exposure of NMDA (20 μM) or a nitric oxide donor, DETA/NO (300 μM) does not produce toxicity. When NMDA and DETA/NO were co-applied, a complete loss of neurons was observed (FIG. 9A). The synergism between NMDA and nitric oxide in producing neurotoxicity was also observed when other nitric oxide donors [DEA/NO (100 μM) and DPT/NO (300 μM)] were used. Previously it has been shown that exposure of neurons to NMDA can generate superoxide. Therefore, when a nitric oxide donor is applied to the cultures treated with NMDA, nitric oxide may react with superoxide to form peroxynitrite intracellularly. It was also determined that DETA/NO and kainate have a synergistic effect in producing neurotoxicity (FIG. 9B).

PQQ is Protective Against Neurotoxicity Induced by Exposure of NMDA Plus DETA/NO

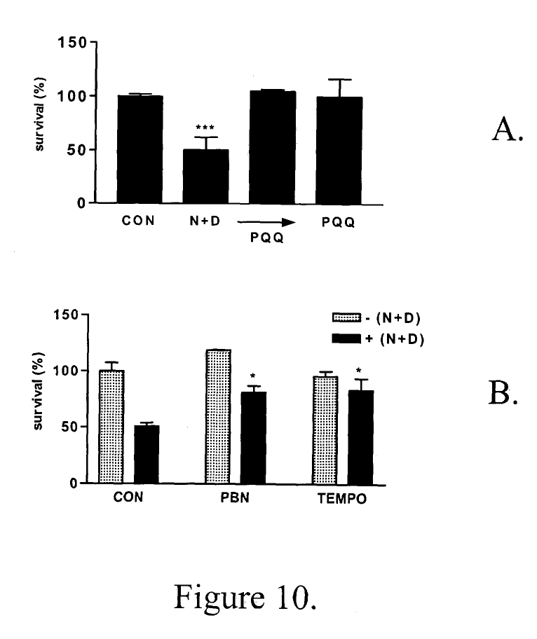

A four-hour exposure to NMDA (20 μM) plus DETA/NO (300 μM) to neurons causes significant (p<0.001) neurotoxicity (FIG. 10A). In four experiments, the neurotoxicity was 52.6±12.1% of control. This toxicity is completely blocked by PQQ at 100 μM (FIG. 10). The free radical spin traps, PBN at 10 mM and TEMPO at 1 mM, also produced complete neuroprotection (FIG. 10B).

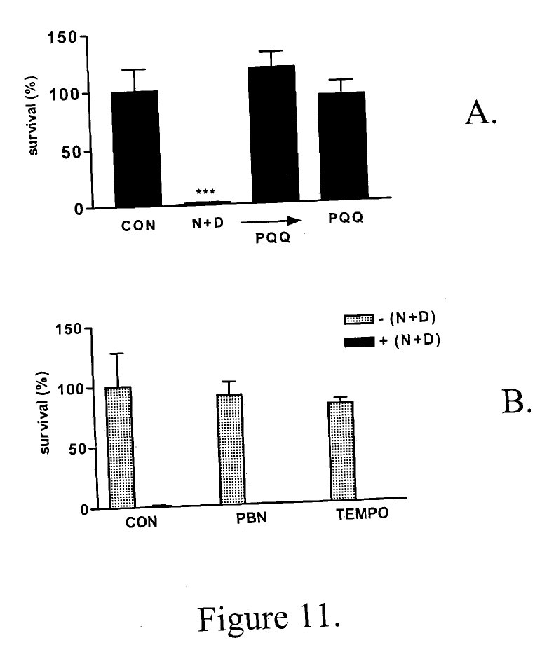

The neurotoxicity induced by NMDA plus DETA/NO at twenty-four hours has also been examined. The NMDA plus DETA/NO-induced toxicity is fully blocked by PQQ (100 μM) (FIG. 11A). However, PBN and TEMPO are not demonstrably neuroprotective (FIG. 11B). These results suggest that, in addition to acting as a free radical scavenger, PQQ may also block downstream signaling events in the cell death pathway directly, and/or by activating gene transcription.

### Example 3

### Pyrroloquinoline Quinone Prevents SIN-1-Induced White Matter Injury

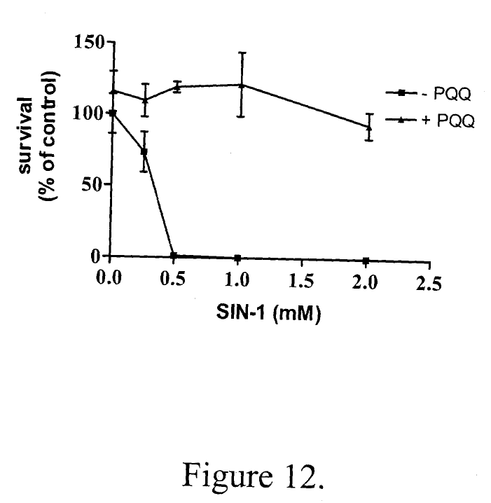

To illustrate the neuroprotective benefits of the invention, PQQ was used to show oligodendrocytes protection from cell toxicity in an in vitro white matter injury assay. Oligodendrocytes were exposed to various concentrations of SIN-1 (0, 0.25, 0.5, 1 and 2 mM) in EBSS for 2 hr with or without PQQ (100 μM), then washed with HBSS containing 0.1% BSA for 2 times. Oligodendrocytes were then returned to the culture medium. The neurotoxicity was examined at 24 hr. The EC₅₀ of SIN-1 toxicity in oligodendrocytes was 345±91 μM. As demonstrated in FIG. 12, PQQ at 100 μM completely blocks oligodendrocyte cell death induced by SIN-1 at various concentrations.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference.

What is claimed is:

1. A method for preventing or inhibiting peroxynitrite-induced neurotoxicity in a mammalian subject in need thereof, comprising administering to said subject an effective amount of a pyrroloquinoline quinone compound, wherein peroxynitrite is not present in toxic levels in said mammalian subject, such that peroxynitrite-induced neurotoxicity is prevented or inhibited.

2. The method of claim 1, wherein said pyrroloquinoline quinone is administered at a concentration between about 1 nM and less than about 100 μM.

3. The method of claim 1, wherein said pyrroloquinoline quinone is administered at a concentration between about 1 μM and about 100 μM.

4. The method of claim 1, wherein said pyrroloquinoline quinone is administered at a concentration between about 100 μM and about 500 μM.

5. The method of claim 1, wherein said subject suffers from or is at risk of amyotrophic lateral sclerosis (ALS), and said method treats neurological manifestations of ALS.

6. The method of claim 1, wherein said pyrroloquinoline quinone compound is formulated in polyethylene glycol or liposomes to enhance central nervous system absorption and efficacy.

7. A method for preventing, inhibiting or reducing neurological damage incident to a stroke in a mammalian subject at risk thereof, comprising administering to said subject an effective amount of a pyrroloquinoline quinone compound prior to a measureable increase in peroxynitrite formation in said mammalian subject, such that neurological damage incident to stroke is prevented, inhibited or reduced.

8. A method for preventing, inhibiting or reducing neurological damage incident to a ALS in a mammalian subject at risk thereof, comprising administering to said subject at risk of or suffering from ALS an effective amount of a pyrroloquinoline quinone compound prior to a measurable increase in peroxynitrite formation in said mammalian subject, such that neurological damage incident to ALS is prevented, inhibited or reduced.

9. A method for treating a chronic neurological disorder in a mammalian subject in need thereof, comprising administering to said subject an effective amount of a pyrroloquinoline quinone compound at a point wherein peroxynitrite is not detectable at toxic levels, such that said chronic neurological disorder is reduced or eliminated.

10. A method for inducing cell toxicity in tissue, comprising contacting said tissue with an effective amount of a pyrroloquinoline quinone compound, wherein said pyrroloquinoline quinone compound is capable of interacting with peroxynitrite, such that neural cell death increases.

11. The method of claim 10, wherein said tissue is neural tissue.

12. The method of claim 11, wherein said neural tissue comprises a malignant cell.

13. A method for preventing or inhibiting peroxynitrite-induced cell death in a mammalian subject at risk of or undergoing an acute traumatic neurological event, comprising administering to said subject an effective amount of a pyrroloquinoline quinone compound at such time wherein peroxynitrite is not detectable at toxic levels, such that peroxynitrite-induced cell death is prevented or inhibited.

14. A method for preventing or inhibiting peroxynitrite-induced neurotoxicity in a mammalian subject in need thereof, comprising administering to said subject an effective amount of a pyrroloquinoline quinone compound, whereby superoxide levels are reduced, such that peroxynitrite-induced neurotoxicity is prevented or inhibited.

15. A method for reducing endogenous peroxynitrite levels in a mammalian subject in need thereof, or in a cell, tissue or organ of said mammalian subject, by administering to said subject or said cell, tissue or organ thereof an effective amount of a pyrroloquinoline quinone compound, such that the endogenous peroxynitrite levels are reduced or at least maintained.

16. A kit for treating or preventing a neurotoxic injury, comprising in one or more containers pyrroloquinoline quinone, a pharmaceutically acceptable carrier, and instructions for use of said kit.

17. A method of treating or preventing a neurotoxic injury, comprising administering to a subject at risk of an imminent neurotoxic injury caused by increased peroxynitrite levels, comprising administering, when peroxynitrite levels are not yet neurotoxic, a combination therapy including a PQQ compound and a second antioxidant, such that said neurotoxic injury is treated or at least partially alleviated.

18. A method for preventing or inhibiting peroxynitrite-induced injury to white matter in a mammalian subject in need thereof, comprising administering to said subject an effective amount of a pyrroloquinoline quinone compound, wherein peroxynitrite is not present in toxic levels in said mammalian subject, such that peroxynitrite-induced injury to white matter is prevented or inhibited.

19. The method of claim 18, wherein said white matter comprises one or more oligodendrocytes.

20. The method of claim 18, wherein said mammalian subject is suffering from or at risk of cerebral palsy or multiple sclerosis.
